(19) United States
(12) Patent Application Publication
(10) Pub. No.: US 2001/0044614 A1
(43) Pub. Date: Nov. 22, 2001

(54) Reducing agents for feminine care products

(76) Inventors: Emmanuelle Cecile Damay, Neenah, WI (US); David Charles Potts, Dunwoody, GA (US); Jack Nelson Lindon, Alpharetta, GA (US); Dmitry Yavich, Appleton, WI (US); Stephen Quirk, Alpharetta, GA (US)

Correspondence Address: Mark E. Fejer, Pauley Petersen Kinne & Fejer, Suite 365, 2800 West Higgins Road, Hoffman Estates, IL, 60195, US

(21) Appl. No.: 09/729,917

(22) Filed: Dec. 4, 2000

Related U.S. Application Data
(60) Provisional application No. 60/171,998, filed on Dec. 23, 1999.

(51) Int. Cl. A61F 13/15

(52) U.S. Cl. 604/385.01; 604/367

(57) ABSTRACT

A personal care absorbent article having at least one nonwoven web material and at least one reducing agent disposed on at least a portion of a surface of the at least one nonwoven web material.

### BACKGROUND OF THE INVENTION

1. Field of the Invention

This invention relates to an absorbent material for absorbing blood-containing fluids. More particularly, this invention relates to an absorbent material for use in personal care absorbent articles, which are particularly adapted for absorbing various blood-containing bodily fluids while providing comfort and fit to the wearer, such as catamenial articles such as sanitary napkins, pads and tampons, wound dressings, and the like. Even more particularly, this invention relates to the use of reducing agents on one or more components, such as the outer cover and the fluid distribution layers of feminine care products, which reducing agents modify the rheology of the mucus component of menstrual fluid, thereby improving the intake rate of menstrual fluid into the sanitary pad or tampon, enabling the reduced mucus component to wick away from the insult point, and reducing premature leakage and cover smearing.

2. General Background

A wide variety of disposable absorbent articles for collecting bodily fluids are known in the art. Commercial absorbent articles include diapers, sanitary napkins, training pants, and incontinent care pads, wound dressings, and the like. Disposable products of this type include some functional elements for receiving, absorbing, and retaining fluids. Typically, such absorbent articles have an absorbent core containing cellulosic fibers, for example, wood pulp fluff, particles of highly absorbent materials, for example, superabsorbents, and an admixture of cellulosic fibers and superabsorbents. Typically, such articles include a fluid-permeable cover sheet or topsheet which typically faces the body of the user, an absorbent core, and a fluid-impermeable backsheet.

Cover sheet materials are utilized for the transport of bodily fluids into the absorbent core of personal care absorbent articles and, thus, materials used for cover sheet applications must manage distinctly different body excretions, depending upon the application and the product type. Some products must manage fluids, such as urine, while others must manage proteinaceous and viscoelastic fluids, such as menstrual discharge and fecal matter. The management of viscoelastic menstrual discharge by feminine care products such as sanitary pads and napkins is exacerbated due to the variations in composition and rheology over a broad range of elasticity. Fluid management in feminine care applications requires control of absorption of bodily fluids, control of fluid retention in the cover, control of stain size and intensity, control of rewet of fluid back to the surface, and control of the release of fluid to the absorbent core.

Menstrual discharges are composed of blood, vaginal or cervical secretions and endometrial tissues. The vaginal secretions are mainly composed of mucins. The proportions of the various components of menstrual fluid vary from woman to woman and from period to period. The proportions of these components also depend upon the age of the woman, the activity of the woman and the method of birth control used by the woman. As a result, the fluid composition can vary from 30 to 70% blood, 10 to 50% cervical secretions, and 0 to 30% endometrial tissues.

Mucin and endometrial tissues are two components that are not easily absorbed into a porous structure made of standard nonwoven materials. These two highly viscous and elastic components are often responsible for cover smearing on a pad, premature leakage (leakage without high content fluid loading in the pad).

There are several factors which influence the flow of liquids in fibrous structures including the geometry of the pore structure in the fabrics, the nature of the solid surface (surface energy, surface charge, etc.), the geometry of the solid surface (surface roughness, grooves, etc.), the chemical/physical treatment of the solid surface, and the chemical nature of the fluid.

In the case of feminine care products such as sanitary pads and napkins, women have come to expect a high level of performance in terms of comfort and fit, retention of fluid, and minimal staining. Of utmost importance, leakage of fluid from the pad onto undergarments is regarded as totally unacceptable.

Improving the performance of feminine care products continues to be a formidable undertaking, although numerous improvements have been made in both their materials and structures. However, solutions addressing the issues arising from the presence of red blood cells in blood or menses in feminine care products, as well as other absorbent materials for handling blood-containing fluids, have not been satisfactorily implemented. It is apparent that a system which effectively handles menses fluids in a manner which addresses the issues set forth hereinabove will not only improve the distribution of incoming fluids by the absorbent material, but will also reduce the tendency toward premature failures of these absorbent articles.

PCT International Publication No. WO 95/19191 teaches superabsorbent materials and compositions containing superabsorbent materials which exhibit enhanced blood absorbency properties for use in disposable, superabsorbent products such feminine hygiene articles and medical articles. There, the superabsorbent material is treated with a polymeric enhancing agent selected from the group consisting of polyglycols, a polycarboxylic acid, a polycarboxylate, a poly(lactone)polyol, a polyamide, a polyamine, a polysulfonic acid, a polysulfonate and combinations thereof.

### SUMMARY OF THE INVENTION

Accordingly, it is one object of this invention to provide a blood-absorbing personal care absorbent article having improved fluid handling, including improved fluid intake and wicking, and reduced staining characteristics.

It is another object of this invention to provide a feminine care absorbent product having improved fluid handling, including improved fluid intake and wicking, and reduced staining characteristics.

It is another object of this invention to provide a method for modifying the rheology of the mucus component of menstrual fluid so as to improve the intake rate of menstrual fluid in feminine care products such as sanitary pads and tampons.

It is another object of this invention to provide a method for modifying menstrual fluid so as to reduce premature leakage and cover smearing in feminine care products such as sanitary pads and napkins.

These and other objects of this invention are addressed by a personal care absorbent article comprising at least one nonwoven web material and at least one reducing agent disposed on at least a portion of the surface of the at least one nonwoven web material.

Without intending to be restricted to a single explanation as to the mechanism of this invention, it is believed that the reducing agent breaks down some critical disulfide intramolecular and/or intermolecular bonds in the mucus glyco-protein or mucin component of the menstrual fluid, thereby significantly decreasing the viscoelasticity of the mucus. Suitable reducing agents comprise a material selected from the group consisting of L-cysteine, thioglycolates, dithiotriacol and combinations and mixtures thereof at a suitable pH. U.S. Pat. No. 6,060,636 teaches alternative means for altering the viscoelastic properties of a viscoelastic composition including the use of protein-cleaving enzymes which act to decrease the size of components of the composition by means of hydrolytic cleavage. The viscoelastic treatments disclosed therein are not reducing agents and there is no chemical reduction occurring as in the instant invention.

Personal care absorbent articles in accordance with this invention typically comprise a fluid pervious cover sheet or body-side liner, a fluid impervious backing sheet, and an absorbent/fluid distribution layer disposed between the top sheet and the backing sheet. The reducing agent utilized in this invention is disposed on at least a portion of the surface of the outer cover or body-side liner. In accordance with one preferred embodiment of this invention, in addition to being disposed on the cover sheet, the at least one reducing agent is also disposed on the distribution layer.

### BRIEF DESCRIPTION OF THE DRAWINGS

These and other objects and features of this invention will be better understood from the following detailed description taken in conjunction with the drawings wherein:

FIG. 1 is a cross-sectional view of a personal care absorbent article in accordance with one embodiment of this invention;

FIG. 2 is a graphical representation showing the reduction of fluid intake time as a result of the application of a reducing agent to the cover sheet or the cover sheet and the distribution layer of a personal care absorbent article;

FIG. 3 is a diagram of a cover sheet for a personal care absorbent article in accordance with one embodiment of this invention;

FIG. 4 is a diagram of a test apparatus for determining the fluid intake time of a fluid into a material; and

FIG. 5 is a diagram showing the effect of treatment using cysteine on a viscoelastic material.

### DESCRIPTION OF PREFERRED EMBODIMENTS DEFINITIONS

As used herein, the term “viscoelastic” means a composition having at least one significant component that is moderately viscous and/or has elastic properties. By “moderately viscous” it is meant that the component has a viscosity of at least that of normal human blood plasma. By “elastic” it is meant that the component has elasticity equal to or greater than normal human blood plasma.

As used herein, the term “reducing agent” refers to a chemical agent that, when an effective amount is contacted by a viscoelastic composition, alters the properties of that viscoelastic composition by decreasing the molecular size of one or more components of the viscoelastic composition by chemical reduction (chemical reaction in which the compound being reduced gains one or more electrons (Hackh's Chemical Dictionary, 3rd Ed.)), i.e. breaking down the component by bond cleavage to produce smaller molecules. Decreasing the molecular size of the one or more components in this manner results in a thinning of the viscoelastic composition, that is a decrease in the viscosity of the viscoelastic composition.

As used herein, the term “nonwoven web” or “fibrous web” refers to any material comprising fibrous or fiber-like elements, usually in a random arrangement, joined by bonding points which stabilize the structure, providing at least some mechanical integrity, which form at least some small pores throughout the length and width thereof between adjacent fiber-like elements. The term also includes individual filaments and strands, yarns or tows as well as foams and films that have been fibrillated, apertured or otherwise treated to provide porosity. “Nonwoven webs” or “fibrous webs” are formed by many processes such as, for example, spunbonding, meltblowing, airlaid and bonded carded processes.

As used herein, the term “spunbonding” refers to a process in which small diameter fibers are formed by extruding molten thermoplastic materials as filaments from a plurality of fine, usually circular capillaries of a spinneret with the diameter of the extruded filaments then being rapidly reduced as, for example, described in U.S. Pat. No. 4,340,563 to Appel et al., U.S. Pat. No. 3,692,618 to Dorschner et al., U.S. Pat. No. 3,802,817 to Matsuki et al., U.S. Pat. No. 3,338,992, U.S. Pat. No. 3,341,394 to Kinney, U.S. Pat. No. 3,502,763 to Hartmann, U.S. Pat. No. 3,502,538 to Levy, and U.S. Pat. No. 3,542,615 to Dobo et al. Spunbond fibers are quenched and generally not tacky when they are deposited onto a collecting surface. Spunbond fibers are generally continuous and have average diameters frequently larger than 7 microns, more particularly, between about 10 and 20 microns.

As used herein, the term “meltblowing” refers to a process in which fibers are formed by extruding a molten thermoplastic material through a plurality of fine, usually circular, die capillaries as molten threads or filaments into converging high velocity, usually heated, gas (for example air) streams which attenuate the filaments of molten thermoplastic material to reduce their diameter, which may be to microfiber diameter. Thereafter, the meltblown fibers are carried by the high velocity gas stream and are deposited on a collecting surface, often while still tacky, to form a web of randomly dispersed meltblown fibers. Such a process is disclosed, for example, by U.S. Pat. No. 3,849,241 to Butin. Meltblown fibers are microfibers which may be continuous or discontinuous and are generally smaller than 10 microns in average diameter.

As used herein, the term “bonded carded” or “bonded carded webs” refers to nonwoven webs formed by carding processes as are known to those skilled in the art and further described, for example, in U.S. Pat. No. 4,488,928 to Alikhan and Schmidt. Typically, carding processes involve starting with a blend of, for example, staple fibers with bonding fibers or other bonding components in a bulky batt that is combed or otherwise treated to provide a generally uniform basis weight. This web is heated or otherwise treated to activate the adhesive component, resulting in an integrated, usually lofty nonwoven material.

As used herein, the term “biconstituent fibers” refers to fibers which have been formed from at least two polymers extruded from the same extruder as a blend. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner.

As used herein, the term “polymer” generally includes, but is not limited to, homopolymers, copolymers, such as for example, block, graft, random and alternating copolymers, terpolymers, etc., and blends and modifications thereof. In addition, unless otherwise specifically limited, the term “polymer” includes all possible geometric configurations of the material. The configurations include, but are not limited to, isotactic, atactic, syndiotactic, and random symmetries.

As used herein, the term “absorbent material” refers to any material having fluid absorption properties.

As used herein, the term “personal care absorbent articles” refers to diapers, training pants, absorbent underpants, adult incontinence products, sanitary wipes and feminine hygiene products such as sanitary napkins, pads and tampons.

As used herein, the term “intake” refers to the ability of an absorbent article to absorb fluid. Intake time is used to assess the quality of absorption with lower intake times denoting materials capable of rapid absorption and higher intake times denoting materials with poorer absorption.

As used herein, the term “stain” refers to fluid, wet or dry, which is present on the top surface, in, or on the bottom surface of a cover material or topsheet of a personal care absorbent article.

“Proteinaceous fluids” refers to a fluid that contains protein or protein breakdown products such as blood or menses. For purposes of evaluation of the material treatment system of this invention, a menstrual simulant was utilized which has similar properties to menstrual discharge.

“High viscoelastic simulant” or “menses simulant” is a material which simulates the viscoelastic and other properties of menses. The first step is preparing a high viscoelastic biological simulant is to prepare 120 mL of ovomucin by separating the yolk and egg whites of about one dozen large eggs, saving the egg white and removing the white strand of egg white. The egg whites are filtered one time using a 2 mm nylon mesh by placing the egg whites on the filter and allowing them to sit on the mesh for 5 minutes while gently moving the egg white on the filter. The material on the top of the filter is referred to as thick egg white. 120 ml of thick egg white is then placed in a 300 ml transfer bag followed by the addition of 80 ml of plasma into the 300 ml transfer bag and gentle mixing of the solution by hand until it looks fairly homogeneous. The solution is placed into a Stomacher mixer (Stomacher 400 Laboratory Blender, Seward Medical, London SE1 1PP UK) at the low setting for 60 seconds. From there, the mixture is placed into a dialysis bag having dialysis clips on each end in a manner which minimizes the amount of air in the bag. The filled bag is weighed (initial wt.), placed into a trough with Superabsorbent Polymer (Favor 880 Stockhausen, Inc. 2401 Doyle Street Greensboro, NC 27406) covering the bag on all sides and refrigerated for 6 hours. Thereafter, the superabsorbent with water is rinsed off and the bag dried thoroughly. The bag is reweighed (weight loss is typically about 46-50 grams), and the volume of fluid after dialysis is measured using a 60 cc syringe. Next, swine blood is centrifuged at 3000 rpm, 20 EC for 30 minutes. The plasma is separated from red blood cells with a disposable pipette. The red blood cells are saved and a 70% mixture of ovomucin/plasma and 30% red blood cells is made. The mixture is gently mixed on a magnetic plate and the resulting solution put into a transfer bag (marking down the volume). Using a syringe, excess air in the bag is removed and the mixture manually gently mixed for 5 minutes. The mixture is then refrigerated for 24 hours before use.

To use the simulant for testing, it is warmed for 10 minutes at 22EC in a water bath before testing. The simulant is manually mixed in the bag for 4 minutes (no visual separation should be seen), the amount needed for testing removed and placed in a beaker. The simulant is then mixed using a magnetic stirrer (on lowest setting) for 5 minutes.

“Low viscoelastic simulant” or “menses simulant” is another material which simulates the viscoelastic and other properties of menses. To prepare the fluid, blood, such as defibrinated swine blood, is separated by centrifuge at 3000 rpm for 30 minutes, although other methods or speeds and times may be used if effective. The plasma is separated and stored separately, the buffy coat removed and discarded, and the packed red blood cells stored separately as well. Eggs, such as jumbo chicken eggs, are separated, the yoke and chalazae discarded, and the egg white retained. The egg white is separated into thick and thin portions by straining the white through a 1000 micron nylon mesh for about three minutes, and the thinner portion discarded. Alternative mesh sizes may be used, and the time or method may be varied provided the viscosity is at least that required. The thick portion of egg white which was retained on the mesh is collected and drawn into 60 cc syringes which are then placed on a programmable syringe pump and the fluid homogenized by expelling and refilling the contents five times. In our case, the amount of homogenization was controlled by the syringe pump rate of about 100 ml/min, and the tubing inside diameter of about 0.12 inches. After homogenizing, the thick egg white has a viscosity of about 20 centipoise at 150 sec-i and it is then centrifuged to remove debris and air bubbles. After centrifuging, 80 mL of the thick homogenized egg white, which contains ovomucin, is added to a 300 cc FENWAL Transfer Pack using a syringe. Then, 60 cc of the swine plasma is added to the transfer pack. The transfer pack is clamped, all air bubbles removed, and placed in a Stomacher lab blender in which it is blended at normal (or medium) speed for about two minutes. The transfer pack is then removed from the blender, 60 cc of swine red blood cells are added, and the contents mixed by hand kneading for about two minutes, or until the contents appear homogeneous. The final mixture has a red blood cell content of about 30 volume percent and generally is at least within the range of 28-32 volume percent for artificial menses. The amount of egg white is about 40 weight percent.

This invention involves the use of reducing agents in personal care absorbent articles and, in particular, feminine care products such as sanitary pads and tampons for modifying the rheology of the mucus component of menstrual fluid. Suitable reducing agents include, but are not limited to, cysteine, thioglycolates, dithiothreitol and combinations thereof, as well as other sulfur-containing thiols materials. Personal care absorbent articles in accordance with this invention typically include a fluid pervious cover sheet 10, a fluid impervious back sheet 11 and an absorbent core 12 disposed between the fluid pervious cover sheet and the fluid impervious back sheet as shown in FIG. 1. Materials used in the production of these personal care absorbent articles include nonwovens which may be produced by any method known to those skilled in the art for producing nonwoven web materials. The fibers from which the nonwoven web materials may be made are produced, for example, by meltblowing or spunbonding processes, including those processes producing bicomponent, biconstituent or polymer blend fibers which are well known in the art. These processes generally use an extruder to supply melted thermoplastic polymer to a spinneret where the polymer is fiberized to yield fibers which may be staple length or longer. The fibers are then drawn, usually pneumatically, and deposited on a moving foraminous mat or belt to form the nonwoven fabric.

Alternatively, the nonwoven web may be a bonded carded web. Bonded carded webs are made from staple fibers, which are usually purchased in bales. The bales are placed in a picker, which separates the fibers. Then, the fibers are sent through a combing or carding unit, which further breaks apart and aligns the staple fibers in the machine direction to form a generally machine direction-oriented fibrous nonwoven web. Once the web is formed, it is then bonded by one or more of several known bonding methods, such as powder bonding wherein a powdered adhesive is distributed through the web and then activated by heating the web and adhesive with hot air or some other heat source, pattern bonding wherein heated calender rolls or ultrasonic bonding equipment are used to bond the fibers together, usually in a localized bond pattern, though the web can be bonded across its entire surface, if so desired, and through-air bonding.

As previously stated, menstrual discharges, in particular the mucins and endometrial tissue components thereof are not readily absorbed into a porous structure made of standard nonwoven materials, the result of which is frequently cover smearing and/or premature leakage. Mucins are glycoproteins that form very long chains with many carbohydrate branches. The physical (mechanical entanglement) and chemical (disulfide bonds, ionic reactions, hydrogen bonding) interactions between these long molecular chains result in the formation of very thick, stringy and viscous fluids called cervical mucus.

The use of L-cysteine, like other reducing agents, to reduce disulfide bonds is well known in the literature. A similar compound, N-acetylcysteine, is used as a mucolytic agent to improve clearance in cystic fibrosis and in cough medications. L-cysteine also plays several critical roles in the body. Its more important roles are protecting cells and cellular components against oxidative stress and in detoxification. L-cysteine is a natural sulphur-containing amino acid derivative found naturally in foods and is a powerful antioxidant. These dual properties help repair oxidative damage in the body. This has made this nutrient of special interest to athletes for some time as heavy exercise increases oxidative damage in the body. The most recent research interests are in connection with AIDS and heart disease. For these reasons, L-cysteine is an amino acid used in some food supplements and drugs.

Chemical alteration of the mucus glycoprotein can produce a thinner fluid that can be easily absorbed in porous nonwoven structures. The method of this invention employs a reducing agent, such as L-cysteine, to break down the long glycoprotein into smaller segments. L-cysteine breaks down critical disulfide bonds in the mucus glycoprotein.

Bench test results indicate that L-cysteine is very effective at thinning the mucus glycoprotein, thereby allowing a rapid intake of very thick mucus-containing fluid. By breaking down some critical disulfide intra and/or intermolecular bonds in the mucus glycoprotein, the L-cysteine significantly decreases the viscoelasticity of the mucus. Thus, the use of this invention can improve the intake rate of menstrual fluid in sanitary pads and tampons, allow the reduced mucus component to wick horizontally instead of staying confined at the insult point, and reduce premature leakage and cover smearing. The use of this invention as a treatment on the cover material and/or on the distribution layer will allow improved absorbency in a feminine care absorbent product. In addition, this invention may be used on a cover material in combination with cover topography and geometry to allow, simultaneously, faster intake rate, better dryness and lower rewet.

One method for determining the effectiveness of a reducing agent in breaking down the disulfide intra and/or intermolecular bonds in the mucus glycoprotein is the use of ANS (8-Anilino-1-naphthalenesulfonate), a substance that fluoresces in a non-aqueous environment but not in an aqueous environment. If the hydrophobic sites of a protein are accessible, then ANS will fluoresce. If the number of hydrophobic sites increases, the fluorescence also increases. An increase of hydrophobic sites indicates that the protein is being broken down. Indirectly, an increase in ANS emission indicates that the mucin protein has been reduced. Such an increase was observed on the addition of cysteine to a mucin-containing solution.

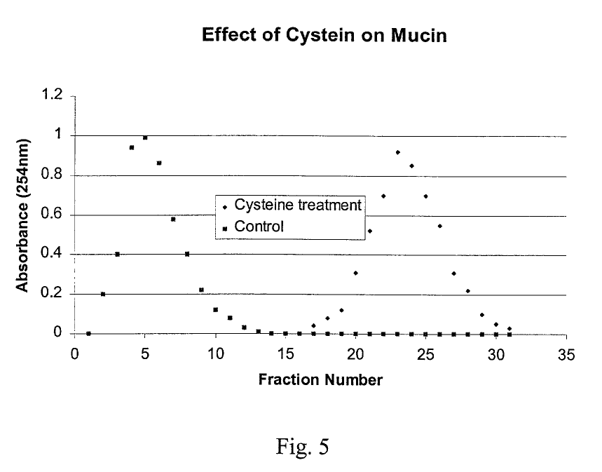

Another method for determining the effectiveness of a reducing agent is with gel permeation chromatography. FIG. 5 shows the results obtained from the treatment of mucin with the reducing agent cysteine. As can be seen, the mucin treated with cysteine was eluted at significantly higher fraction numbers than the control, mucin with no treatment. In gel permeation chromatography, higher fraction numbers correspond to smaller sized molecules and smaller sized molecules of mucin translates into a material in which viscoelasticity has been reduced.

The direct addition of 2% by volume L-cysteine to menstrual fluid simulants produces rapid decrease in the viscoelasticity of the fluid. Tables 1 and 2 hereinbelow show the effects of 2% by volume L-cysteine on both high and low viscoelasticity menses simulants, five (5) minutes after addition, using a Vilastics III rheometer available from Vilastic Scientific, Inc. located in Austin, Tex. operating at a frequency of 0.1 Hz.

TABLE 1
Reduction of Viscoelastic Components of High Viscoelastic Menses
Simulant After 5 Minutes Incubation with 2% by Volume L-cysteine

| Sample | Viscosity (Poise) | Elasticity (Poise) |
|---|---|---|
| Control (undiluted) | 0.8 | 0.75 |
| 2% Cysteine | 0.15 | 0.04 |

TABLE 2
Reduction of Viscoelastic Components of Low Viscoelastic Menses
Simulant After 5 Minutes Incubation With 2% by Volume L-cysteine

| Sample | Viscosity (Poise) | Elasticity (Poise) |
|---|---|---|
| Control (undiluted) | 0.32 | 0.23 |
| 2% Cysteine | 0.14 | 0.05 |

To rapidly reduce the viscoelasticity of mucus, a concentration of 2% by volume cysteine in the fluid is preferred. To achieve high concentration in use on very light weight thin cover materials, a relatively large amount of cysteine should be added onto the fabric. Different treatment approaches have been employed to achieve a desired add-on of cysteine on a creped spunbond cover. Creped spunbond was chosen because the topography of the creped spunbond allows for higher add-on.

A first method consists of solubilizing cysteine in an aqueous solution and then spraying the solution on the creped spunbond cover material. As L-cysteine is not soluble in a neutral aqueous solution, cysteine hydrochloride monohydrate was used. Cysteine hydrochloride is soluble in water, but the solution obtained is highly acidic (pH=1.4). Neutralization of the pH is obtained by adding sodium hydroxide to the solution. The creped spunbond is sprayed with a 10% cysteine solution until a 250% wet pick up, resulting in 25% by weight cysteine on the fabric. The fabric is then air dried and treated with a 10% PLURONIC® F105 solution available from BASF Corporation until a 20% wet pick up, resulting in a 2% PLURONIC F105 by weight on the fabric.

### EXAMPLE 1

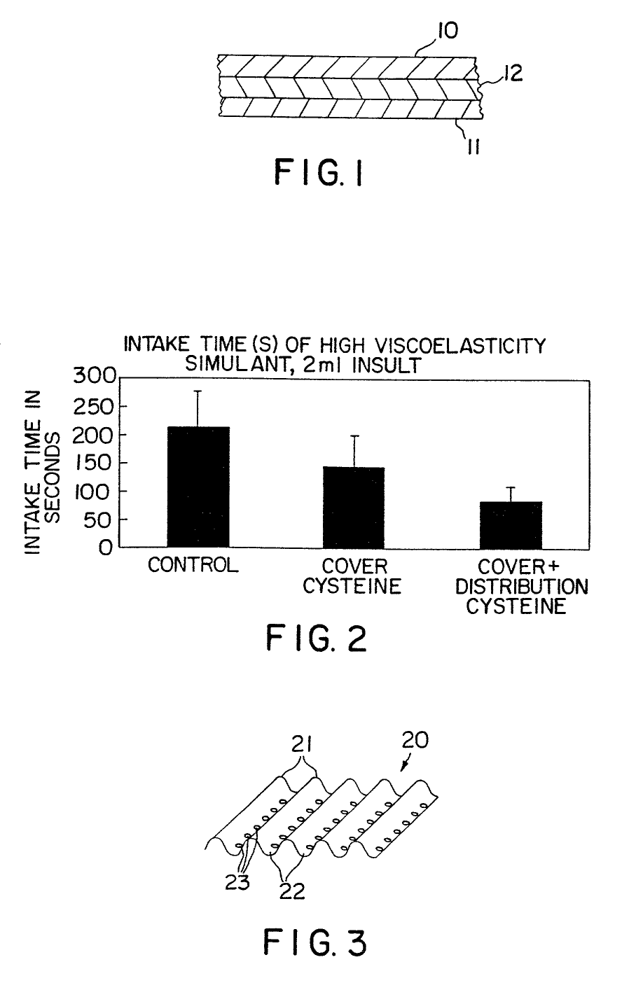

Creped spunbond polypropylene, 5 denier per fiber (dpf), a basis weight of about 0.4 osy, 30% crepe, 12″×8″ hand sheets (about 1.4 gr./hand sheet untreated) was first weighed. A 10% by weight cysteine solution (40 gr. L-(+)-cysteine hydrochloride, Monohydrate from JT Baker, Product #G121-05, Lot #34583, 360 gr. distilled water, and 9.95 gr. of NaOH for neutralization of the pH to 7) was sprayed using an atomizer on both sides of the creped spunbond until a 250% add-on was achieved. The wet weight after cysteine spraying was about 4.9 gr. based upon an initial weight of 1.4 gr. The hand sheets were then air dried for 2 hours, and oven dried for 30 minutes at 70EC. The dry weight after cysteine treatment was about 1.75 g. The cysteine add-on weight was 0.25 gr. of cysteine per 1 gr. of spunbond material (25%). The creped spunbond cover treated with 25% by weight cysteine showed some statistically significant improvement in intake rate for the high viscoelasticity simulant when compared to the control creped spunbond cover treated with PLURONIC only (See FIG. 2.) However, there was a lot of variability in intake times due to non-uniformity of the creped spunbond fabric and non-uniformity of the treatment on the fabric. It was noted that orientation of the creped spunbond also influences the intake time measured with the rate block test.

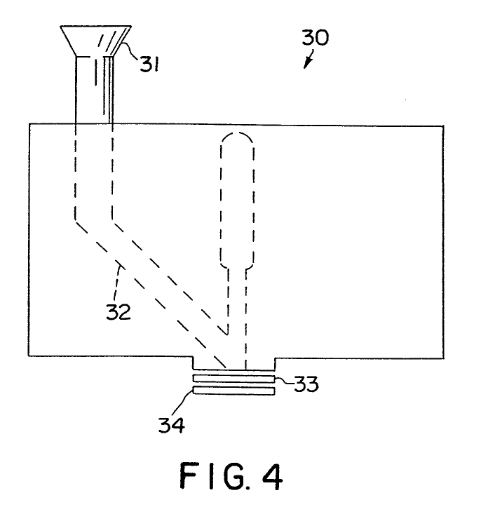

This test is used to determine the intake time of a known quantity of fluid into a material and/or material system. The test apparatus consists of a rate block 30 as shown in FIG. 4. A 4″×4″ piece of each of the absorbents 34 and cover 33 are die cut. The specific covers are described in the specific examples. The absorbent used for these studies was standard and consisted of a top piece (closest to the cover) of a 90% Coosa 0054/10% Hoechst-Celanese T-255 binder, 100 gsm, 0.1 g/cc airlaid web and a bottom piece which was a 90% Coosa/10% Hoechst-Celanese T-255 binder, 200 gsm, 0.2 g/cc airlaid web. The cover 33 was placed over the two pieces of absorbent 34 and the rate block 30 was placed on top of the two materials. 2 mL of a menses simulant was delivered into the test apparatus funnel 31 and a timer started. The fluid moved from the funnel 31 into a channel 32 where it was delivered to the material or material system. The timer was stopped when all the fluid was absorbed into the material or material system as observed from the chamber in the test apparatus. The intake time for a known quantity of known fluid was recorded for a given material or material system. This value is a measure of a material or material systems absorbency. Typically, five to ten repetitions were performed, and average intake time was determined.

A second method consists of suspending the cysteine particles in a thickener. The treatment for this thickener consists of saturating the cover materials with the cysteine/thickener mixture, and then passing the material over a slot vacuum to suck off any excess fluid. This method produces a uniform treatment and 200% and 300% by weight wet add-on can be achieved. Because fine particles allow a more uniform coating, the particles are sieved. Particles under 200 microns produce better results and, thus, are preferred. This method allows for formation of an homogeneous mixture of the cysteine and there is no sedimentation of insoluble particles as a result of which the treatment can be uniform; a high percentage add-on (between about 50 and 75% by weight dry add-on) can be achieved; and it promotes the binding of the cysteine particles to the spunbond fibers.

Various thickeners have been tried: sodium alginate high (14,000 centipoise of 2% solution at 25EC), medium (3,500 centipoise) and low (250 centipoise) viscosity ranges and guar gum. Other additives like wetting agents (such as PLURONIC P105) and softeners (silk proteins, aloe) have been mixed with the thickener. Creped covers coated with cysteine using a thickener method in accordance with one embodiment of this invention improved intake time of fluid that contains a high percentage of mucus. Bench tests using a highly viscoelastic biological simulant comprising only a mixture of ovomucin and plasma at body temperature accentuated the effect of cysteine: for the control, absorption of a 1 ml insult of this fluid takes more than five minutes as opposed to the cysteine treated cover where absorption of a 1 ml insult took only one minute (See Table 3).

### EXAMPLE 2

Creped spunbond polypropylene, 5 denier per fiber (dpf), a basis weight of about 0.4 osy, 30% crepe, 12″×8″ handsheets (about 1.4 gr./handsheet untreated) (polymer E5D47 8% AMPACET® 41438, E5D47 is available from Shell Chemical Company and AMPACET is available from Ampacet Corporation, Mt. Vernon, N.Y.) was first weighed. The creped spunbond material was then dipped until saturated in a treatment solution comprising 3% Alginic Acid Sodium Salt from Macrocytis pyrifera, Medium viscosity from Sigma Aldrich, St. Louis, Mo., Product No. A-2033 Lot 94H0284, 1% PLURONIC P105 from BASF (Germany), 20% L-cysteine also from Sigma Aldrich, Product No. C-7755 Lot 58H0441 and 76% distilled water. The handsheets were then put on a slot vacuum for aspiration of excess fluid until 380% by weight wet add on was reached. The handsheets were then air dried. The dry weight add on of cysteine was equal to 66%.

Table 3 shows a comparison of fluid intake rate for a control spunbond cover treated with 1% PLURONIC P105 and a cover treated with cysteine in a thickener using the procedure of this Example 2.

TABLE 3
Fluid Intake Rates

| Sample | 1^{st} Insult | 2^{nd} Insult |
|---|---|---|
| Control | 112 sec. | 1640 sec. |
| Cysteine Treated Cover | 51 sec. | 104 sec. |

The tests were conducted in an environmental chamber set at 37EC and 80% humidity. The covers were placed on the top of a 250 gsm airlaid material in the environmental chamber and tested with a high viscoelasticity simulant without red blood cells at 37EC. Fluid was placed on the covers and the time for the first ml of fluid to go into the material was measured. After 30 seconds, the time for a second ml of fluid to go into the material was measured.

FIG. 2 also shows the reduction of fluid intake time by cysteine treatment on a cover material alone and on a cover/distribution layer composite. Although treatment of only the cover material results in a reduction in fluid intake time compared to a control, the fluid intake time is reduced even more when both the cover and the distribution layer are treated with cysteine.

A preferred cover design that combines the topography, the geometry and chemical treatment in accordance with this invention is shown in FIG. 3. The cover 20 comprises a plurality of alternating peaks 21 and valleys 22. In accordance with the embodiment shown, the cover forms a plurality of apertures 23 in valleys 22. The topography of this cover design allows isolation of the fluid from the body. The fluid flows into the valleys, and the ridges close to the body stay dry. The geometry allows faster fluid intake through the apertures. The apertures are not a problem for rewet because they are located in the valleys far from the body. Disposition of the cysteine treatment in the valleys allows thinning of the fluid and consequently faster intake and better wicking in the underneath distribution layer.

The previously described reducing agent application methods generally involve physical coating of the nonwoven materials with the reducing agent of choice. However, reduction of the viscoelasticity of mucin is also achieved by chemical bonding of the reducing moiety to the surface of the polymeric fibers comprising the nonwoven materials. Application of the reducing agent to the polymer surface in accordance with this embodiment can be accomplished by ionizing a sulfur-containing compound in the presence of the polymeric fibers resulting in bonding of the ionic sulfur component of the ionized compound to the carbon backbone of the polymer of the polymeric fibers. Hydrogen ions are then brought into contact with the modified polymer resulting in replacement of any groups attached to the sulfur with hydrogen. The resulting surface of the polymeric fibers has the ability to reduce the viscoelasticity of mucin.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

We claim:

1. A personal care absorbent article comprising:
at least one nonwoven web material; and
at least one reducing agent disposed at least one of on and in at least a portion of said at least one nonwoven web material.

2. A personal care absorbent article in accordance with claim 1, wherein said at least one reducing agent is applied as a coating to said at least one nonwoven web material.

3. A personal care absorbent article in accordance with claim 1, wherein said at least one reducing agent is chemically bonded to a surface of at least a portion of a plurality of polymeric fibers of said nonwoven web material.

4. A personal care absorbent article in accordance with claim 1, wherein said reducing agent is a sulfur-containing reducing agent.

5. An article in accordance with claim 4, wherein said reducing agent comprises a material selected from the group consisting of cysteine, thioglycolates, dithiothreitol and combinations and mixtures thereof.

6. An article in accordance with claim 5, wherein said reducing agent is L-cysteine.

7. An article in accordance with claim 5, wherein said reducing agent is a thioglycolate.

8. An article in accordance with claim 1, wherein said at least one nonwoven web material is a body-side liner of said personal care absorbent article.

9. An article in accordance with claim 1, wherein said at least one nonwoven web material is a fluid distribution layer of said personal care absorbent article.

10. An article in accordance with claim 1, wherein said at least one nonwoven web material is a material selected from the group consisting of spunbonded, meltblown, airlaid, and bonded carded web and combinations thereof.

11. An article in accordance with claim 1, wherein said reducing agent is present in an amount corresponding to at least about 1% by volume of a menses fluid contacting said article.

12. An article in accordance with claim 8 further comprising at least one nonwoven fluid distribution layer.

13. An article in accordance with claim 1, wherein said at least one nonwoven web material is a corrugated material comprising a plurality of alternating peaks and valleys.

14. An article in accordance with claim 13, wherein said reducing agent is disposed in said valleys.

15. An article in accordance with claim 13, wherein said corrugated material forms a plurality of openings in said valleys.

16. An article in accordance with claim 1, wherein said at least one reducing agent is combined with a thickener to form a reducing agent/thickener mixture.

17. A method for improving the intake rate of menstrual fluid by a feminine care absorbent article comprising the steps of:
forming a feminine care absorbent article comprising an absorbent material and an outer cover disposed over at least a portion of said absorbent material; and
applying at least one reducing agent suitable for breaking down at least one of intramolecular and intermolecular disulfide bonds in said menstrual fluid to said outer cover.

18. A method in accordance with claim 17, wherein said at least one reducing agent is deposited on a fluid distribution layer of said feminine care absorbent article.

19. A method in accordance with claim 17, wherein said at least one reducing agent is a material selected from the group consisting of cysteine, thioglycolates, dithiothreitol and combinations thereof.

20. A method in accordance with claim 17, wherein said at least one reducing agent is added to said outer cover in an amount corresponding to about 2% by volume of said menstrual fluid.

21. A method in accordance with claim 17, wherein said outer cover is a creped material.

22. A method in accordance with claim 17, wherein said at least one reducing agent is deposited on said outer cover by spraying.

23. A method in accordance with claim 17, wherein said at least one reducing agent is combined with a thickener, forming a reducing agent/thickener mixture, and said outer cover is saturated with said reducing agent/thickener mixture.

24. A method in accordance with claim 23, wherein said thickener comprises a material selected from the group consisting of alginates, guar gum and combinations and mixtures thereof.

25. A method in accordance with claim 23, wherein said thickener is sodium alginate.

26. A method in accordance with claim 23, wherein an additive selected from the group consisting of wetting agents, softeners, and combinations and mixtures thereof is mixed with said thickener.

27. A method in accordance with claim 21, wherein said creped material is a spunbond.

28. A method in accordance with claim 21, wherein said creped material comprises a plurality of alternating peaks and valleys and said reducing agent is deposited in said valleys.

29. A method in accordance with claim 17, wherein said at least one reducing agent is chemically bonded to a surface of at least a portion of a plurality said polymeric fibers of said outer cover.

30. A method in accordance with claim 17, wherein said feminine care absorbent article is a tampon.

31. A method in accordance with claim 17, wherein said feminine care absorbent article is a sanitary pad.

32. A tampon comprising:
at least one nonwoven web material; and
at least one reducing agent disposed at least one of on and in at least a portion of said at least one nonwoven web material.
